(19) United States
(12) Patent Application Publication
(10) Pub. No.: US 2002/0146467 A1
(43) Pub. Date: Oct. 10, 2002

(54) Herbal composition for the prevention and treatment of dementia

(76) Inventors: Kyu Yong Jung, Kyunggi-do (KR); Kyung Soo Keum, Daegu (KR)

Correspondence Address: FINNEGAN, HENDERSON, FARABOW, GARRETT &, DUNNER LLP, 1300 I STREET, NW, WASHINGTON, DC, 20005, US

(21) Appl. No.: 09/850,269

(22) Filed: May 8, 2001

(30) Foreign Application Priority Data
Feb. 8, 2001 (KR) 2001-6177

(51) Int. Cl. A61K 35/78

(52) U.S. Cl. 424/725; 424/757

(57) ABSTRACT

The present invention relates to herbal composition for the prevention and treatment of dementia comprising Polygoni multiflori Radix (Polygonum multiflorum Thunberg), instead of ginseng, Polygalae Radix (Polygala tatarinowi Regel), Caryophylli Flos (Eugeni caryophyllata Thunb), and Zingiberis Rhizoma Crudus (Zingiber officinale Rosc) in the conventional herbal composition to maximize anti-demetia effect with minimized amount. And thus, the herbal composition of the present invention provides maximized activity toward dementia, especially senile dementia, with not only minimized side effects, but also improved pharmacological and clinical activities compared to the conventional products.

### BACKGROUND OF THE INVENTION

1. Field of the Invention

The present invention relates to herbal composition for the prevention and treatment of dementia comprising Polygoni multiflori Radix (Polygonum multiflorum Thunberg), instead of ginseng, Polygalae Radix (Polygala tatarinowi Regel), Caryophylli Flos (Eugeni caryophyllata Thunb), and Zingiberis Rhizoma Crudus (Zingiber officinale Rosc) in the conventional herbal composition to maximize anti-demetia effect with minimized amount. And thus, the herbal composition of the present invention provides maximized activity toward dementia, especially senile dementia, with not only minimized side effects, but also improved pharmacological and clinical activities compared to the conventional products.

2. Description of the Related Art

Recently the magnitude of the dementia problems as well as senile dementia has been rapidly increased. To date, dementia has no known prevention or cure except several treatment drugs showing extremely limited effects toward dementia, although there have been intensive studies in the development of drugs or foods to prevent or cure dementia in eastern or western countries. Still intensive researches have focused on senile dementia and Alzheimer's disease (AD), which refer to “progressive loss of cognition and intellectual abilities, in the biochemical, genetic and medical aspects. More specifically, main symptoms of dementia include cognitive function impairment and various mental disorders in language, judgment and perceptive vasospastic ability as well as serious difficulty in acquisition of new technologies. Personality changes and emotional restlessness soon become apparent and ultimately to death.

Dementia, which adversely affects the intrinsic activity of cerebrum, is a peculiar symptom associated with the fundamental disorders of brain induced by various factors. For example, the brain was grossly shrunken in size and the expansion of the ventricle in most cases due to loss of cerebral parenchyma, a large number of cerebral cortex cells, Purkinje cells in cerebellium or eukaryotic cells at spinal cord become disappeared. The causes of these symptoms are unknown but they have been noticed to have significant relations with the level of neurotransmitter acetylcholine (ACh).

Commercially available anti-dementia agents include Cognex and Done Pezyl, which are known to inhibit against the activity of acetylcholine esterase (AChE) acting mainly on the central nervous system, thus increasing the level of neurotransmitter acetylcholine for the prevention and treatment of dementia.

However, a majority of the conventional anti-dementia agents may produce serious cholinergic effects in the peripheral nerve with an extremely short half-life and severe side effects such as hepatotoxicity (ref: Br. J. Psychiatry, 138, 46, 1981). Further, Cognex (9-amino-1,2,3,4-tetrahydroacridine, THA), which have been widely used for the treatment of dementia, is effective significantly in enhancement of cognitive ability in AD patients during oral administration (ref: N, Engl. J. Med., 315, 1241, 1986) but many adverse reactions such as tremor, dizziness and cytotoxicity have still encountered.

On the other hand, Korean Patent Publication No. 1999-85202 which is the corresponding patent of U.S. Pat. No. 6,010,702 discloses herbal extracts having ginseng as a major component to inhibit against AChE activity with less severe side effects.

These herbal extracts show some effects toward dementia but they are not reliably effective. And it is only expected that the action mechanism of the herbal extracts is different from that of other dementia agents, although the herbal extracts showed some effects for senile dementia. In particular, the use of ginseng, which is a major component of the conventional herbal composition, leads to some adverse reactions such as palpitation of the heart for patients with cardiovascular diseases and functional homeostatic imbalance. Ginseng is widely known to recover intrinsic energy but it will be prohibited from using ginseng as an anti-dementia agent by the following reasons reported in the literature.

Ginseng may deprave the symptoms of patients who suffer from hypernoia, nervous prostration, hypertension or arteriosclerosis due to its side effects. And further, it is well known in oriental medicine theory that ginseng should not be used to patients suffering from pulmonary tuberculosis, asthma and cough (Bonchogujin, Tree and Soil 1999; Professional Handbood of Complementary and Alternative medicine; Charles W. Fetrow, Springhous, 1999) and recently, there are clinical reports suspecting its use.

Furthermore, compositions of such herbal extracts are relatively administered with excess amount, resulting in unexpected side effects and toxicities therefrom.

### SUMMARY OF THE INVENTION

Even though the herbal extracts show less side effects compared to other drugs, there are not enough clinical studies about exact therapeutic efficacy and other side effects. Therefore, it is still strongly demanded to develop more effective herbal extracts. In order to supply herbal extracts to general publics as health foods for the prevention and treatment of dementia, it will be more important to minimize both dose amount and side effects.

Under this circumstance, the inventor has developed the first herbal extract composition registered in the US (U.S. Pat. No. 6,010,072) for the treatment of dementia and further made intensive studies to develop a novel drug having an inhibitory action on AChE with little adverse reaction and under the judgment that a drug containing some herbs may lessen any adverse reactions, screened some herbal components having an inhibitory action on AChE. As a result of repeated the efficacy screening and toxicity tests in a composition where some supplemental herbs are added to the conventional herbal extracts having an inhibitory action on ACHE, it has been confirmed that the herbal composition of this invention has superior effects in the prevention and treatment of dementia by improving blood-circulation disturbance and facilitating the glucose metabolism in the neurons. In consequence, the inventor has completed this invention.

Therefore, an object of the present invention is to provide superior herbal composition for the prevention and treatment of dementia having improved pharmacological and clinical actions compared to the conventional herbal composition by replacing ginseng with Polygoni multiflori Radix (Polygonum multiflorum Thunberg) and additionally adding Polygalae Radix (Polygala tatarinowi Regel), Caryophylli Flos (Eugenia caryophyllata Thunb), and Zingiberis Rhizoma Crudus (Zingiber officinale Rosc) to the conventional composition.

### DETAILED DESCRIPTION OF THE INVENTION

This invention is characterized by an extract composition comprising 10-30 weight parts of Polygoni multiflori Radix (Polygonum multiflorum Thunberg), 1-10 weight parts of Polygalae Radix (Polygala tatarinowi Regel), 1-10 weight parts of Caryophylli Flos (Eugenia caryophyllata Thunb), and 1-10 weight parts of Zingiberis Rhizoma Crudus (Zingiber officinale Rosc) in the herbal composition comprising Arisaematis Rhizoma (Arisaema japonicum BL.), Gastrodiae Rhizoma (Gastrodia elata BL.), Acorus Gramninecus (Acorus gramineus Soland), Ostericum Koreanum (Curcuma longa L.), Bambusae Caulis In Taeniam (Phyllostachys nigra Munro var), Bombycis Corpus (Bombyx mori L.), Ponciri Fructus (Poncirus trifoliata Raf), Hoelen (Poria cocos Wolf), Pinelliae Tuber (Pinellia ternata Breit), Aurantii nobilis Pericarpium (Citrus unshiu Marcor) and Glycyrrhizae Radix (Glycyrrhiza uralensis Fisch).

The present invention is described in detail as set forth hereunder:

The invention is characterized by the extract composition comprising 10-30 weight parts of Polygoni multiflori Radix, 1-10 weight parts of Polygalae Radix, 1-10 weight parts of Caryophylli Flos, and 1-10 weight parts of Zingiberis Rhizoma Crudus, 1-10 weight parts of Arisaematis Rhizoma, 1-10 weight parts of Gastrodiae Rhizoma, 1-8 weight parts of Acorus Graminecus, 1-10 weight parts of Ostericum Koreanum, 1-8 weight parts of Bambusae Caulis In Taeniam, 1-8 weight parts of Bombycis Corpus, 1-8 weight parts of Ponciri Fructus, 1-10 weight parts of Hoelen, 1-10 weight parts of Pinelliae Tuber, 1-10 weight parts of Aurantii nobilis Pericarpium and 1-5 weight parts of Glycyrrhizae Radix by a typical extraction process.

These extracts may be used as herbal drug composition or supplementary drinks because they have shown excellent effects for the prevention and treatment of dementia. The herbal composition may be formulated as pellets, extract liquid preparations, granules, infused preparations, decocted preparations, tablets, capsules or parenteral preparations dissolved in distilled water, if deemed necessary, but the most preferably pellets.

One cannot ascertain the mechanism of each component of herbals to inhibit against AChE in their own nature but as a result of repeated comparative studies on some herbals having various composition, a herbal having the above herbal components and composition ratio has been confirmed to have the most optimal therapeutic efficacy.

According to the oriental medicinal theory, even if herbal drug compositions are similar, expected efficiencies and toxicities of each composition are quite different. Accordingly, the herbal composition of the present invention is comprised to enhance the therapeutic efficacy of the prevention and treatment of dementia and minimize the toxicity, thus providing the maximized effects.

The conventional herbal composition has recognized some disadvantage in, that the excessive use of drug components has caused adverse reactions and toxicities, when clinical doses are administered. Since the use of ginseng, one of the active ingredients, is associated with some adverse reactions such as palpitation, blood-circulation disorder in patients with cardiovascular disease such as hypertension, and pulmonary tuberculosis, asthma and cough, the overall homeostatic imbalance in the body may easily occur. It is, therefore, preferred not to add ginseng to the composition of this invention. At this point it is quite difficult to screen some of novel active ingredients useful for the prevention and treatment of dementia, even though it is most preferred that the pharmacological action and clinical efficacy of the composition be sustained with little side effects. Therefore, the use of effective active ingredients in the composition represents the combination of entirely different components.

This invention is characterized by the novel use of Polygoni multiflori Radix instead of ginseng used in the conventional arts, thus enhancing the pharmacological and clinical effectiveness for the prevention and treatment of dementia with less side effects.

The active ingredients of Polygoni multiflori Radix according to this invention include cresophanol, rein, emodin, physhion and their glycosides; it has been reported that these ingredients acting on intrinsic energy and blood of human are effective in promoting hepatic function, extra strength and blood nourishment. Unlike ginseng, Polygoni multiflori Radix has reportedly anti-aging properties such as better renal function, recovery of hair function and better body condition, as shown in several literatures (Chinese Pharmaceutics, Shinminkyo, Namsandang, 1986; Modern Herbal Science, Society for Herbal Medicine, Hakenun Sa, 1992). Specifically, Polygoni multiflori Radix is mainly indicated in the treatment of senile neurological disease and blood-circulation disorder such as cardiac pain, palsy and epilepsy. It is deemed that the adequate combination of Polygoni multiflori Radix with other herbal components may be effective in the prevention and treatment of dementia.

An object of this invention is to maximize the anti-dementia effect using Polygoni multiflori Radix instead of ginseng based upon several conventional literatures and clinical rationale, while ensuring more excellent anti-dementia effect with its interaction with other herbal components by taking advantage of hepatic enhancement and better blood circulation manifested by Polygoni multiflori Radix. However, excessive use of Polygoni multiflori Radix may adversely affect the cardio- and digestive systems; in case of using a small amount, the anti-dementia effect may not be expected.

In addition, the composition of this invention include another novel component “Caryophylli Flos” in a small amount, known as a leading drug in the oriental field, together with Polygoni multiflori Radix. As a result, it has proven that the cerebral action has been further improved. In the aspect of pharmacology, the use of Caryophylli Flos may induce other active ingredients contained in the composition, resulting in better enhancement of cerebral action in terms of pharmacological and clinical action. However, a small amount of Caryophylli Flos may reduce the anti-dementia effect but in case of using an excessive amount, vomiting and abdominal pain may occur.

Meantime, Acori graminei Rhizoma is indicated in the treatment of cerebral disorder such as dementia and amnesia. Since the use of Acori graminei Rhizoma itself in the conventional composition may not manifest sufficiently the anti-dementia effect, the inventor et al. has pursued another herbal component which can be mixed with Acori graminei Rhizoma. In consequence the inventor et al. has found out that the concurrent use of Acori graminei Rhizoma with Polygalae Radix exhibits the pharmacological synergy effect based upon the oriental theory of Chinese Pharmaceutics. With the addition of small amounts of Polygalae Radix with Acori graminei Rhizoma, the composition of this invention has successfully demonstrated better pharmacological action. However, the excessive use of Polygalae Radix with Acori graminei Rhizoma may be associated with blood-circulation disturbance and indigestion but in case of using a small amount of Polygalae Radix with Acori graminei Rhizoma, the anti-dementia effect may be reduced in the absence of any synergy effect with Acori graminei Rhizoma.

Further, the use of Zingiberis Rhizoma Crudus in the composition of this invention can alleviate various toxicities associated with such ingredients as Pinelliae Tuber, etc. In particular, Pinelliae Tuber comprises a lot of toxic alkaloids including ephedrine, its long-term or excessive use may produce blood-circulation disorder and neurological excitation. Thus the composition of this invention can maximize the anti-dementia effect through the removal of various toxicities using Pinelliae Tuber. However, a small amount of Pinelliae Tuber may cause any toxic effect but in case of excessive use of Pinelliae Tuber, the clinical efficacy in the composition of this invention may be decreased.

Especially, the appropriate use of various herbal components according to this invention can enhance the cerebral blood flow in the body, thus supplying sufficient amount of glucose to cerebral neurons as energy source to facilitate the glucose metabolism in the neurons.

The enhanced energy metabolism has contributed much to sustaining the homeostasis of cerebral neurons, while preventing the degeneration of cerebral neurons by inducing the secretion and separation of neurotransmitter.

Therefore, the pharmacological mechanism of this invention may be significantly effective in the prevention and treatment of dementia characterized by the gradual cognitive loss.

As described above, the anti-dementia composition of this invention contains Polygoni multiflori Radix as an active ingredient, as well as other herbal components such as Polygalae Radix, Caryophylli Flos and Zingiberis Rhizoma Crudus. With the various content ratios, the composition of this invention with better pharmacological effect and less side effects is quite effective in the prevention and treatment of dementia. In addition, the composition of this invention is an epoch-making anti-dementia drug and superior health food, when it is used for the general public during the long-term period.

Each component in the herbal composition of this invention is extracted from water and alcohol. For example, said mixture is extracted with water after heating for 4-10 hrs or with ethanol for 2-5 hrs, filtered, and evaporated to give a powder which is further formulated by adding additives to obtain one of appropriated preparations.

When the herbal composition of this invention is orally administered as pellets for the desired therapeutic effect to patients who suffer from senile dementia, an extract powder for an adult (60 kg) may be administered 2-4 times daily with a dose of 5-15 g, preferably 7.5-12.5 g each time.

The administration dose has been determined with consideration of toxicities and side effects. When the excess amount of the herbal composition is administered for long term, it has not shown severe side effects and provided superior therapeutic effects to small administration.

When the herbal extracts having ginseng as an effective component is administered to patients, it requires special care because most of patients are suffering from hypertension. However, the herbal composition of this invention can be safely administered to patients suffering from hypertension, other blood-circulation disorder or hepatic disease with much less side effects and thus, it is highly expected to be used as functional health food.

The construction and effect of this invention is explained in detail based on the following manufacturing examples and experimental examples as set forth hereunder. However, these examples and experimental examples are the ones to further understand this invention and thus, the scope of this invention is not limited by these examples and experimental examples in any respects.

### EXAMPLE 1

A mixture consisting of Polygoni multiflori Radix (20g), Polygalae Radix (3 g), Caryophylli Flos (1 g), Arisaematis Rhizoma (7 g), Gastrodiae Rhizoma (8 g), Acorus Graminecus (4 g), Ostericum Koreanum (8 g), Bambusae Caulis In Taeniam (3 g), Bombycis Corpus (3 g), Ponciri Fructus (³ g), Hoelen (8 g), Pinelliae Tuber (8 g), Aurantii nobilis Pericarpium (7 g), Glycyrrhizae Radix (2 g), and Zingiberis Rhizoma Crudus (4 g) was placed in 0.8L of water, heated slowly and refluxed for 4-10 hrs. Then, a liquid-phase solution was filtered through filter paper and filtrate was dried over freeze dryer to obtain a light-brown powder (10.0 g).

### EXAMPLE 2-7

A powder composition comprising the same components in Example 1 was prepared to have the following component ratios shown in Table 1.

TABLE 1

| | Used amount (g) | | | | | | |
|---|---|---|---|---|---|---|---|
| Example | 1 | 2 | 3 | 4 | 5 | 6 | 7 |
| Polygoni multiflori | 20 | 15 | 18 | 20 | 20 | 30 | 30 |
| Radix | | | | | | | |
| Polygalae Radix | 3 | 4 | 2 | 5 | 4 | 4 | 3 |
| Caryophylli Flos | 1 | 1.5 | 2 | 3 | 3 | 1 | 1 |
| Arisaematis Rhizoma | 7 | 8 | 5 | 10 | 8 | 8 | 8 |
| Gastrodiae Rhizoma | 8 | 7 | 8 | 8 | 8 | 8 | 8 |
| Acorus Graminecus | 4 | 4 | 3 | 3 | 3 | 2 | 3 |
| Ostericum Koreanum | 8 | 4 | 8 | 8 | 10 | 10 | 5 |
| Bambusae Caulis In | 3 | 4 | 4 | 4 | 3 | 3 | 3 |
| Taeniam | | | | | | | |
| Bombycis Corpus | 3 | 4 | 4 | 4 | 3 | 3 | 2 |
| Ponciri Fructus | 3 | 4 | 4 | 4 | 3 | 3 | 2 |
| Hoelen | 8 | 7 | 8 | 8 | 10 | 10 | 10 |
| Pinelliae Tuber | 8 | 7 | 10 | 10 | 8 | 8 | 8 |
| Aurantii nobilis | 7 | 8 | 10 | 10 | 10 | 8 | 8 |
| Pericarpium | | | | | | | |
| Glycyrrhizae Radix | 2 | 2 | 2 | 2 | 2 | 2 | 2 |
| Zingiberis Rhizoma | 4 | 10 | 2 | 8 | 8 | 5 | 10 |
| Crudus | | | | | | | |
| Water | 0.8 L | 1L | 0.8L | 1.5L | — | — | — |
| Ethanol | — | — | — | — | 0.8L | 1.5L | 1.5L |
| Yield | 10.0 | 9.9 | 10.2 | 12.0 | 10.8 | 12.5 | 13.0 |

### Comparative Example 1

A mixture consisting of ginseng (50 g), Arisaematis Rhizoma (15 g), Gastrodiae Rhizoma (15 g), Acorus Graminecus (10 g), Ostericum Koreanum 10 g, Bambusae Caulis In Taeniam (10 g), Bombycis Corpus (10 g), Ponciri Fructus (10 g), Hoelen (10 g), Pinelliae Tuber (10 g) and Aurantii nobilis Pericarpium (6 g) and Glycyrrhizae Radix (6 g) was placed in 1L of water, heated slowly and refluxed for 6-8 hrs. Then, a liquid-phase solution was filtered through a filter paper and filtrate was completely dried over a freeze dryer to obtain a brown powder (9.4 g).

### Comparative Example

A herbal mixture containing the same components and composition ratio as Example 1 was extracted 1L of ethanol for 3 hrs. Then, a liquid-phase solution was filtered through a filter paper and filtrate was completely dried over a freeze dryer to obtain a brown powder (3.0 g).

### Experimental Example 1: Inhibitory Activity Against AChE

To evaluate the inhibitory effect of an extract of this invention on ACHE, each extract powder (0.025, 0.05 and 0.1 mg/ml) prepared from Example 1 was added to an enzyme (1,000 units) extracted from an electric eel (AChE: Sigma Cat. No. C-2888) dissolved in 1 ml PBS (phosphate buffer: 0.1M, pH 7.4). Based on a each AChE activity was measured by Ellman's coupled assay using an UV-visible spectrophotometer at 412 nm using a velocity constant factors of enzyme, Km (Micaelis constant), Vmax (maximum speed) and 5,5-dithio-bis-(2-nitrobenzoic acid: DTNB) as a coupling agent. As shown in the following table 1, the inhibitory effect of an extract of this invention on AChE showed that at doses of 0.05 mg/ml and 0.1 mg/ml, the extract of this invention induced decrease in the enzymatic activity by 30.3% and 80.4%, respectively.

TABLE 2

| | |
|---|---|
| Amount of herbal | Loss of enzymatic activity (%) |

| (mg/ml) | Example 1 | Comparative Example 1 |
|---|---|---|
| 0 | 0 | 0 |
| 0.025 | 15.2 | 13.6 |
| 0.05 | 30.3 | 21.0 |
| 0.1 | 80.4 | 59.8 |

It can be safely said that the inhibitory activity (80.4%) of the herbal extract of this invention on AChE is quite remarkable, in consideration of the fact that THA (brandname: Cognex) exhibits about an inhibitory action on AChE by 40% at maximum (ref: Keio J. Med., 36, 381, 1987), and the conventional extracts of U.S. Pat. No. 6,010,702 exhibits 59.8% (Comparative Example 1).

### Experimental Example 2: Inhibitory Activity Against AChE

Through in vivo study to evaluate the inhibitory effect of an extract of this invention on AChE, 20-week-old rats were divided into 2 groups (experimental and control groups) consisting of 7 individuals each prior to commencement of administration. A solution containing 0.5 g of powder, so prepared from Example 1, dissolved in 100 ml distilled water was orally administered to the treatment group at a daily dose of 3 ml for consecutive days, while no medication was given to the control group. After 10 days, the brain of rats was removed to weigh the total weight of brain. Then, 5 ml PBS (0.1 M, pH 7.4) was added to the brain, crushed completely, and stirred slowly for 3-5 hrs. 2 ml PBS (0.1M, pH 7.4) was further added to the solution of brain cell for stirring, centrifuged (Hettich Rotina 48R) at 1,000 rpm at 40 for 10 minutes centrifuge and purified by a filter (CAMEO 25ES nitrocellulose pore size: 0.45 mm)

The AChE activity of rat brain was measured by Ellman's coupled assay (ref: Ellman, G. L., Biochem. Pharmacol., 7, 88, 1961) using an UV-visible spectrophotometer at 412 nm. Hence, the enzymatic reaction rate on all substrates at the intervals of time was calculated by Michaelis-Menten equation. The purified brain was incubated in a 1 ml quartz cuvette containing 790 μl of PBS (1.0M, pH 7.3), 60 μl substrate in 5 ml solution (ATcH: acethylthiocoline) and 120 μl DTNB (dithionitrobenzoic acid) in 5 mM solution as a coupling agent for about 3 minutes and then, each 10 μl of brain solution extracted from the treatment and control groups was added to the quartz cuvette for assessment of ACHE activity, as shown in the following table 3.

TABLE 3

| | Mean initial rate of | Loss of AChE |
|---|---|---|
| Classification | AChE(AU/s) | activity (%) |
| Normal rats | 3.100 × 10⁻³ | 0 |
| Rats with oral administration | 2.042 × 10⁻³ | 34.1 |
| of herbal extracts (3 ml/day) | | |

From the above in vivo test, it was noted that when the herbal extract of this invention was administered to rats for 10 consecutive days, the ACHE activity was reduced by 34.1% compared with rats having no medication. This is much more improved than the activity (23.1%) of Comparative Example 1. Thus, it has proven that the herbal extract of this invention has an improved inhibitory action on AChE.

### Experimental Example 3: Comparison of Neurontransmitter Acetylcholine Concentration

Female Sprague-Dawley rats (pyrogen test free) at three weeks of age were purchased and housed at 23-25□ in a light/dark cycle to grow 20-week-old rats (250-350 g) for use in this study. The herbal extract prepared from Example 3 of this invention was orally administered to each experimental rats consisting of 7 individuals for 10 days in the same manner as Experimental Example 2. After 5 and 10 days of administration, all rats were necropsied and the weight of brain was measured. The brain cells were homogenized by 10ml PBS (phosphate buffer: 0.1M, pH 7.4) containing 0.2% tripton X-100 and centrifuged (1,000 rpm) at 4□ to isolate acetylcholine from the brain cell solution. The acetylcholine, so isolated, was assayed by HPLC equipped with an electrode detector. Some standard graphs of acetylcholine at accurate concentrations were prepared to analyze each sample of 20 μl extracted from the rat brain under the same conditions (flow rate: 10 ml/min, detection scope: 3,9062nA, solvent: 0.1M PBS at pH 7.4). When each group consisting of 7 rats was given an herbal extract to their brains at the intervals of time to compare their level of acetylcholine, so biochemically isolated, with that of normal rats, the results were shown in the following table 4.

TABLE 4

| | Amount of | Increasing rate of | No. of |
|---|---|---|---|
| Day | acetylcholine(μM) | acetylcholine(%) | rats |
| 0 | 107.7 | 0 | 7 |
| (normal rats) | | | |
| 5 | 165.1 | 53.3 | 7 |
| 10 | 241.2 | 123.9 | 7 |

The above results indicated that the actual concentration of neurontransmitter acetylcholine was increased in rats with the herbal extract of this invention, thus reflecting the actual inhibitory effect of such herbal extract on AChE in the body. The concentration of acetylcholine was increased by 53.3% and 123.9% after 5-day and 10-day administration of the herbal extract, respectively. The herbal extract of this invention has more 5-fold, and 2-fold potent inhibitory action on AChE than some other natural products disclosed in the Japanese Patent No. 25760 and the composition of Comparative Example 2, while having at least 2-fold potent inhibitory action on AChE than synthetic anti-dementia agents for the prevention and treatment of dementia (ref: Keio J. Med., 36, 381, 1987). In particular, the naturally occurring herbals used for this studies, which have been clinically used for several thousand years as other purposes of use, has little adverse reactions which have encountered in the medication using the conventional drugs (ref: Experimental Example 11).

### Experimental Example 4: Pellets and its Efficacy Screening

1 g of a dried pellet form prepared from the mixing of 5 g of honey with 100 g of herbal powder, so prepared from Example 4, was dissolved in water (10 ml) every day for its oral administration to 20-week-old Sprague-Dawley rats for 10 consecutive days. After 10 days, the activity of AChE and acetylcholine level in brain was measured in the same manner as Experimental Examples 2 and 3. The results of this experiment revealed that the pellet form had the same inhibitory activity against AChE with increasing concentration of acetylcholine, as in Experimental Examples 2 and 3.

### Experimental Example 5: Powders and its Efficacy Screening

100 g of herbal powder, so prepared from Example 5, was finely pulverized, passed through No. 18 sieve (850 μg), followed by the addition of lactose (200 g) as a diluent to prepare a powder form. Each 0.7 g of the powder form was dissolved in water (10 ml) every day for its oral administration to 20-week-old Spague-Dawley rats for 10 consecutive days. After 10 days, the activity of ACHE and acetylcholine level in brain was measured in the same manner as Experimental Examples 2 and 3. The results of this experiment revealed that the powder form had the same inhibitory action on AChE with increasing concentration of acetylcholine, as in Experimental Examples 2 and 3.

### Experimental Example 6: Tablets and its Efficacy Screening

100 g of herbal powder, so prepared from Example 6, was mixed with 25 g of lactose and 5 g of starch and with the addition of talc (5 g), the mixture was formulated by a tabletting machine to prepare a film-coated tablet. Each 0.7 g of the tablet form was dissolved in water (10 ml) every day for its oral administration to 20-week-old Sprague-Dawley rats for 10 consecutive days. After 10 days, the activity of ACHE and acetylcholine level in brain was measured in the same manner as Experimental Examples 2 and 3. The results of this experiment revealed that the tablet form had the same inhibitory action on AChE with increasing concentration of acetylcholine, as in Experimental Examples 2 and 3.

### Experimental Example 7: Granules and its Efficacy Screening

100 g of herbal powder, so prepared from Example 7, was mixed with 25 g of lactose and 5 g of starch, passed through a sieve (No. 12-45) to prepare a granule form. 1.0 g of the granule form was dissolved in water (10 ml) every day for its oral administration to 20-week-old Sprague-Dawley rats for 10 consecutive days. After 10 days, the activity of ACHE and acetylcholine level in brain was measured in the same manner as Experimental Examples 2 and 3. The results of this experiment revealed that the granule form had the same inhibitory action on ACHE with increasing concentration of acetylcholine, as in Experimental Examples 2 and 3.

### Experimental Example 8: Capsules and its Efficacy Screening

100 g of herbal powder, so prepared from Example 1, was filled into a capsule (No. 3, 0.3 ml). Each 0.7 g of the capsule form was dissolved in water (10 ml) every day for its oral administration to 20-week-old Sprague-Dawley rats for 10 consecutive days. After 10 days, the activity of AChE and acetylcholine level in brain was measured in the same manner as Experimental Examples 2 and 3. The results of this experiment revealed that the capsule form had the same inhibitory action on AChE with increasing concentration of acetylcholine, as in Experimental Examples 2 and 3.

### Experimental Example 9: Infused Preparations and its Efficacy Screening

A herbal mixture having the same components and composition ratio as Example 1 was finely pulverized and with the addition of purified water (200 ml), precipitated for 3 hrs. A thermally-purified water (700 ml) was added and mixed to the resulting solution several times, cooled and filtered by a cotton. 50 g of honey as a cordial was further added to the solution. Each 3 ml of the percolated preparations was orally administered to 20-week-old Sprague-Dawley rats for 10 consecutive days. After 10 days, the activity of ACHE and acetylcholine level in brain was measured in the same manner as Experimental Examples 2 and 3. The results of this experiment revealed that the percolated preparations had the same inhibitory action on AChE with increasing concentration of acetylcholine, as in Experimental Examples 2 and 3.

### Experimental Example 10: Decocted Preparations and its Efficacy Screening

Purified water (900 ml) was added and mixed several times to a herbal mixture having the same components and composition ratio as Example 1, heated for more than 30 min and filtered off in warm state. 50 g of honey were added to the resulting solution as a flavoring agent. Each 3 ml of the boiled agent was orally administered to 20-week-old Sprague-Dawley rats for 10 consecutive days. After 10 days, the activity of ACHE and acetylcholine level in brain was measured in the same manner as Experimental Examples 2 and 3. The results of this experiment revealed that the percolated preparations had the same inhibitory action on AChE with increasing concentration of acetylcholine, as in Experimental Examples 2and 3.

### Experimental Example 11: Liver function Test of Rats in Serum

20-week-old Sprague-Dawley rats were divided into a placebo group (8 rats) and a Experimental group (9 rats). 500 mg of herbal powder, so prepared from Example 1, was dissolved in purified water (1000 ml) and then, each 3 ml of the solution was orally administered to the treatment group at 10 o'clock every morning for 10 consecutive days. After 10 days, the animals were sacrificed to collect the blood samples. The blood samples were centrifuged at 1500 rpm at 4□ for 10 min to separate the sera. Then, the liver function test on such sera was performed according to the conventional method. As revealed in the following table 5, it was noted that both groups had nearly similar levels in AST, ALT, ALP and BUN, thus reflecting that the herbal of this invention had no primary hepatotoxicity. Especially, the herbal extract of this invention showed much lower hepatotoxicity than that of Comparative Example 1.

TABLE 4
Concentration of various parameters in blood

| | AST | ALT | ALP | BUN | |
|---|---|---|---|---|---|
| Group | (IU/l) | (IU/l) | (IU/l) | (IU/l) | n |
| Placebo | 171.3 ± 26.6 | 56.4 ± 14.8 | 433.3 ± 181.3 | 21.2 ± 3.0 | 8 |
| Control | 135.1 ± 28.2 | 56.8 ± 11.4 | 430.1 ± 89.3 | 21.5 ± 1.9 | 9 |
| Experi- | 112.2 ± 38.1 | 58.1 ± 14.8 | 421.3 ± 101.1 | 22.3 ± 2.3 | 9 |
| mental | | | | | |

As described above, the herbal composition of the present invention for the prevention and treatment of dementia comprises Polygoni multiflori Radix as a major component instead of ginseng in the conventional herbal composition and further Polygalae Radix, Caryophylli Flos, and Zingiberis Rhizoma Crudus of which components are not added in the conventional composition. This novel composition shows excellent inhibitory activity against dementia without side effects and toxicities.

Therefore, large amount of the herbal composition of the present invention with an appropriate ratio of each component can be administered for long period due to no side effects and toxicities therefrom and also it can be applied as nutritional supplement drinks to anybody to prevent and cure dementia.

What is claimed is:

1. Herbal extracts for the prevention and treatment of dementia comprising Arisaematis Rhizoma, Gastrodiae Rhizoma, Acorus Graminecus, Ostericum Koreanum, Bambusae Caulis In Taeniam, Bombycis Corpus, Ponciri Fructus, Hoelen, Pinelliae Tuber, Aurantii nobilis Pericarpium and Glycyrrhizae Radix, wherein the herbal extracts comprising 10-30 weight parts of Polygoni multiflori Radix, 1-10 weight parts of Polygalae Radix, 1-10 weight parts of Caryophylli Flos, and 1-10 weight parts of Zingiberis Rhizoma Crudus, 1-10 weight parts of Arisaematis Rhizoma, 1-10 weight parts of Gastrodiae Rhizoma, 1-8 weight parts of Acorus Graminecus, 1-10 weight parts of Ostericum Koreanum, 1-8 weight parts of Bambusae Caulis In Taeniam, 1-8 weight parts of Bombycis Corpus, 1-8 weight parts of Ponciri Fructus, 1-10 weight parts of Hoelen, 1-10 weight parts of Pinelliae Tuber, 1-10 weight parts of Aurantii nobilis Pericarpium and 1-5 weight parts of Glycyrrhizae Radix are extracted by the conventional extraction.

2. The herbal extracts in according to claim 1, wherein said herbal extracts are extracted by the conventional extraction using water and alcohol.

3. A herbal composition containing said herbal extracts of claim 1 or claim 2 as effective components for the prevention and treatment of dementia.

4. The herbal composition in according to claim 3, wherein it is formulated as various dosage forms such as pellets, extract liquid preparations, powders, granules, infused preparations, decocted preparations, tablets, capsules and parenternal preparations.

5. Nutritional supplement food containing said herbal extracts of claim 1 or claim 2 as effective components for the prevention and treatment of dementia.

6. Then nutritional supplement food in according to claim 5, wherein it is formulated as various dosage forms such as pellets, extract liquid preparations, powders, granules, infused preparations, decocted preparations, tablets, capsules and parenternal preparations.
